(19) United States
(12) Patent Application Publication
(10) Pub. No.: US 2023/0002736 A1
(43) Pub. Date: Jan. 5, 2023

(54) ENDODERM DIFFERENTIATION FROM PLURIPOTENT STEM CELL LINES

(71) Applicant: THE REGENTS OF ATHE UNIVERSITY OF CALIFORNIA, Oakland, CA (US)

(72) Inventor: Aras N. Mattis, San Francisco, CA (US)

(21) Appl. No.: 17/783,574

(22) PCT Filed: Dec. 11, 2020
(86) PCT No.: PCT/US2020/064424

§ 371 Date: Jun. 8, 2022

Related U.S. Application Data
(60) Provisional application No. 62/947,308, filed on Dec. 12, 2019.

(51) Int. Cl. C12N 5/071

(52) U.S. Cl. CPC C12N 5/0672; C12N 2501/16; C12N 2506/45

(57) ABSTRACT

Provided are methods for inducing differentiation of pluripotent stem cells, e.g., induced pluripotent stem cells, to form definitive endoderm. Also provided are methods for producing more committed endodermal cells such as hepatoblasts or hepatocytes.

### CROSS-REFERENCE TO RELATED APPLICATIONS

This application claims the priority benefit under 35 U.S.C. § 119(e) of U.S. Provisional Patent Application No. 62/947,308, filed Dec. 12, 2019, which is incorporated herein by reference in its entirety.

### STATEMENT OF GOVERNMENT SUPPORT

This invention was made with government support under grant no. K08 DK098270 awarded by The National Institutes of Health. The government has certain rights in the invention.

### FIELD

The disclosure relates to the fields of embryogenesis and medicine in providing materials and methods for the differentiation of cells into endodermal lineages.

### BACKGROUND

Induced pluripotent stem cells (iPSCs) provide an important tool for the generation of large numbers of terminally or partially differentiated cells including insulin-responsive beta pancreatic cells or metabolically functional hepatocyte-like cells from patients. Hepatocyte-like cells are further significant as they can be used in the characterization of liver diseases and metabolic pathways. The most popular method of differentiating iPSCs to hepatocyte-like cells or lung or pancreatic beta cells is via developmental cues through an endoderm intermediate. Most iPSC clones fail to differentiate into endoderm, however, with induction resulting in apoptosis. Thus, studies of iPSC-derived endoderm cell lines reported to date have been limited to the iPSCs lines that are known to differentiate efficiently. Even under the most ideal conditions, traditional methods only work on a fraction of stem cell lines with more than three quarters of cell lines undergoing cell death or only small patches of cells surviving. In fact, it is considered dogma in the field of endoderm differentiation that there are PSCs that consistently fail to differentiate to endoderm.

In view of the foregoing observations, it is apparent that that there is a need for materials and methods for promoting the induced differentiation of pluripotent stem cells, such as induced pluripotent stem cells, to definitive endoderm cells. The need extends to promotion of further differentiation of the definitive endoderm cells into more committed cell fates such as hepatoblasts and hepatocytes.

### SUMMARY

The disclosure provides methods that significantly improve pluripotent stem cell (PSC) differentiation to endoderm making it possible to differentiate (convert) nearly any PSC to endoderm. The disclosure provides methods that significantly improve the efficiency of differentiating pluripotent stem cells (PSCs), such as induced pluripotent stem cells (iPSCs) or embryonic stem cells (ESCs), into endoderm lineage cells and beyond. In addition to increasing the efficiency of endoderm differentiation of iPSCs, the disclosed methods increase throughput to accommodate culturing in 96-well plates or smaller formats. The improved differentiation protocol includes Activin A as a primary driver of differentiation from PSCs to endoderm by inducing nodal pathways leading to definitive endoderm. The disclosed methods also include doxycycline, which was found to inhibit apoptosis and lead to the proliferation of cells. In some embodiments, the methods comprise an optimized protocol (Mattis protocol 1, i.e., MP1) that consistently provides endoderm from 3 to 7 days.

In order to improve this technology, genes and small molecule compounds were screened and doxycycline, a small molecule, was discovered that, when used in conjunction with MP1 or other commercial differentiation kits, was routinely able to rescue nearly all cell lines from cell death, leading to efficient differentiation with increased proliferation and decreased apoptosis, producing sheets of monolayers in all of the PSCs that were available for testing. This method was able to efficiently produce endoderm even from cell lines that others in the field had disqualified as incapable of forming endoderm. During the development of this project, multiple new induced pluripotent stem cell (iPSC) lines were created that are expected to be useful in the transplant setting, either as iPSCs or after differentiation to an appropriate tissue type, including the cell lines AG02261c-1, AG12107c-Y, AG16104-5, AG06103A-3, GM03529-3, AG02602A, AG09022B, AG16102A, AG16146, GM01680B, GM01863A, GM03524, GM03525A, GM04260C, GM10423, GM20266, GM09093, AS4484, AS4485, AS7017, AS7191, AS7192, and AG16086-1 and their derived subclones. In addition, several new cell lines were developed, at least in part for experimental purposes, in conjunction with this project, including AG06103A GFP-cyMyc-C3+, AS7017-5 GFP-cMYC, and AS7017-5 GFP-BCL-XL.

The differentiation methods disclosed herein provide a unique combination of compounds (Mattis Protocol 1) for differentiation of PSCs, or embryonic stem cells, into endoderm over a 7-day period. The data disclosed herein show that the addition of the small molecule doxycycline, either with or without Y-27632 (or any other rho kinase pathway inhibitor) in combination, significantly improves PSC differentiation into endoderm, including within cell lines thought to be resistant to differentiation to endoderm. The induced pluripotent stem cell (iPSC) lines disclosed herein were produced from cells of patients, including aged, apparently healthy patients, and may represent healthier human tissue as a starting point with possibly more fit genomes.

We tested the protocol disclosed herein using 32 iPSC lines from 16 donors and found consistent formation of complete sheets of endoderm in 29 lines (90%) within 3 to 7 days. Endoderm cells generated by the disclosed methods achieve similar transcriptomic profiles, positive immunohistochemical staining for FOXA2, HNF16, CXCR4, and SOX17, and the ability to be further differentiated into hepatocyte-like cells, cholangiocyte-like cells, or pancreatic Beta cells. Not only do the disclosed methods improve the efficiency of making monolayer sheets of endoderm, they significantly improve the number of cell lines that will form endoderm. The experimental data disclosed herein demonstrate that the disclosed protocol with the addition of doxycycline provides an optimal protocol for the generation of endoderm from iPSCs. We further show that the generated endoderm is similar to the endoderm resulting from application of the inefficient and laborious traditional protocols, as revealed by RNA Sequencing and immunohistochemistry. Also disclosed are experiments investigating the molecular mechanisms by which doxycycline drives and significantly enhances the formation of endoderm. Thus, disclosed herein are methods collectively establishing a simple, efficient and scalable protocol for the creation of endodermal cells, e.g., hepatocyte-like cells, from nearly any iPSC, providing a simple and cost-effective approach to the generation of a variety of endodermal cells for use, e.g., in transplantation.

In one aspect, the disclosure provides a method of inducing differentiation of a pluripotent stem cell (PSC) to a definitive endoderm cell comprising incubating the PSC in cell culture media supplemented with Activin A and an antibiotic, thereby producing a definitive endoderm cell. In some embodiments, the PSC is an induced pluripotent stem cell (iPSC). In some embodiments, the cell culture media comprises an inhibitor of caspase 3 cleavage, such as wherein the inhibitor of caspase 3 cleavage is doxycycline. In some embodiments, the method further comprises contacting the PSC with a Matrigel-coated surface. In some embodiments, the PSC is incubated in cell culture media for about 3 to 7 days, such as wherein the PSC is incubated in cell culture media for 7 days. In some embodiments, the cell culture media is RPMI media supplemented with Gem21 NeuroPlex without insulin at lx concentration according to the supplier's recommendation (Gemini Bio-Products catalog #400-962), Glutagro at 1× concentration according to the supplier's recommendation (Corning, Manassas, Va., catalog #25-015-CI) or at about 2 mM, MEM non-essential amino acids at 1× concentration according to the supplier's recommendation (Gibco, catalog #11-140-050), about 0.5 mM sodium butyrate (Sigma Aldrich, catalog #B5887), about 100 ng/mL Activin A (Peprotech, Rocky Hill, N.J., catalog #120-14E), and less than 10 μg/ml, e.g., about 1-3 OA doxycycline (Acros Organics catalog #446061000). In some embodiments, the method further comprises adding about 2% KnockOut Serum Replacement (KSR; Gibco Catalog #10828028), about 10 ng/mL CHIR-99021 (Selleckchem catalog #S2924), about 10 ng/mL PI-103 (Selleckchem catalog #S1038), about 10 ng/mL BMP4 (Peprotech catalog #120-05ET),and about 20 ng/mL FGF2 (Peprotech catalog #100-18B) on day 1 of incubation. In some embodiments, the method further comprises adding about 1% KSR, about 10 ng/mL PI-103, about 10 ng/mL BMP4, and about 20 ng/mL FGF2 on day 2. In some embodiments, the method further comprises adding about 0.2% KSR and about 10 ng/mL PI-103 on day 3. In some embodiments, the antibiotic is doxycycline (Acros Organics catalog #446061000), Gentamicin (Sigma Aldrich, cat. no. G1264), minocycline (Sigma Aldrich, cat. no. M9511), demeclocycline (Sigma Aldrich, cat. no. D6140), methacycline (Sigma Aldrich, cat no. 37906), KB-R7943 (Sigma Aldrich, cat. no. K4144), or Ruthenium Red (Sigma Aldrich, cat. no. 84071). In some embodiments, the antibiotic is doxycycline or Gentamicin, such as wherein the antibiotic is doxycycline, e.g., wherein the concentration of doxycycline is less than 10.0 μg/mL e.g., 0.1-3.0 μg/mL. In some embodiments, the cell culture media further comprises Y-27632 (Aoex Bio, cat. no. B1293). In some embodiments, the Matrigel-coated surface is the surface of a cell culture plate or a cell culture well.

In another aspect, the disclosure provides an optimized and efficient protocol for the induction of stem cells to endoderm using doxycycline with or without Y-27632. Doxycycline is started the day prior to induction and can be typically continued throughout endoderm induction. In another aspect gentamycin can be used in place of or in combination with doxycycline with or without Y-27632 to enhance the survival of cells in the presence of Activin A. The use of doxycycline or gentamycin improves the final stem cell-derived cell line such as an iPSC-derived hepatocyte (as measured by RNA Sequencing of gene expression across different cell lines).

In another aspect the protocol uses knockout serum replacement (KSR) to enhance survival of cells. During the use of KSR, the small molecule PI-103 can be added to inhibit insulin signaling. The protocol can use FGF2 and BMP4 to enhance the survival during the first two days. Overall the protocol allows for an efficient way to make monolayers or three-dimensional organoids of endoderm that can be pushed into monolayers or three dimensional endoderm-derived tissues. In another aspect, the disclosure provides an improved method of performing fluorescence-activated cell sorting (FACS) or any other method of cell isolation of stem cells in particular as related to the survival as single cells to grow-out to colonies in the presence of doxycycline and/or Y-27632. The addition of these compounds when cells are dissociated increases their survival on replating or when they are placed back into optimal growth conditions. We also note that efficient expansion of cells using this method enables them to proliferate extensively allowing for replating of the cells with a two to four fold expansion. Furthermore this increased proliferation also allows the final product to show improved consistency as measured by gene expression by RNA Sequencing.

In accordance with the foregoing aspects, the disclosure provides a method of inducing differentiation of a pluripotent stem cell (PSC) to a definitive endoderm cell comprising: (a) contacting a PSC with a Matrigel-coated surface; (b) incubating the PSC for about seven days in RPMI media supplemented with Gem21 NeuroPlex without insulin at 1× concentration, Glutagro at 1× concentration or about 2 mM, non-essential amino acids at 1× concentration, about 0.5 mM sodium butyrate, about 100 ng/mL Activin A, and 1-3 μM doxycycline; (c) adding about 2% KnockOut Serum Replacement (KSR), about 10 ng/mL CHIR-99021, about 10 ng/mL PI-103, about 10 ng/mL BMP4,and about 20 ng/mL FGF2 on day 1; (d) adding about 1% KSR, about 10 ng/mL PI-103, about 10 ng/mL BMP4, and about 20 ng/mL FGF2 on day 2; and (e) adding about 0.2% KSR and about 10 ng/mL PI-103 on day 3; wherein definitive endoderm cells are present in the culture on about day 7. In some embodiments, the media is supplemented with 1 μM doxycycline.

Another aspect is directed to a method of inducing differentiation of a definitive endoderm cell to a hepatoblast comprising (a) contacting a definitive endoderm cell with cell culture media comprising Matrigel, wherein the cell culture media is supplemented with Gem21 NeuroPlex without insulin, Glutagro, NEAA, monothioglycerol, human insulin and dexamethasone; and (b) incubating the definitive endoderm cell in the cell culture media for about five days, thereby producing a hepatoblast. In some embodiments, the cell culture media is Iscove's modified Dublecco's medium. In some embodiments, the cell culture media comprises Gem21 NeuroPlex without insulin at 1× concentration, about 1× concentration Glutagro, about lx concentration non-essential amino acids, about 0.3 mM monothioglycerol, about 5 μg/mL human insulin, about 100 nM dexamethasone, about 10 ng/mL Fibroblast Growth Factor 2 and about 20 ng/mL Bone Morphogenetic Protein 4.

Yet another aspect of the disclosure is a method of inducing differentiation of a hepatoblast to a hepatocyte comprising: (a) contacting the hepatoblast for about 5 days with about 10 ng/mL Fibroblast Growth Factor 2, about 20 ng/mL Bone Morphogenetic Protein 4, and about 20 ng/mL human Hepatocyte Growth Factor; and (b) incubating the hepatoblast in Lonza Hepatocyte Culture media BulletKit comprising about 20 ng/mL Hepatocyte Growth Factor and about 20 ng/mL Oncostatin M for about five days, wherein the Lonza Hepatocyte Culture media BulletKit lacked Epidermal Growth Factor, thereby producing a hepatocyte.

Other features and advantages of the disclosure will become apparent from the following detailed description, including the drawing. It should be understood, however, that the detailed description and the specific examples, while indicating embodiments, are provided for illustration only, because various changes and modifications within the spirit and scope of the disclosure will become apparent to those skilled in the art from the detailed description.

### BRIEF DESCRIPTION OF THE DRAWINGS

FIG. 1. Doxycycline Rescues Induction to Endoderm. A: Schematic of C-MYC or BCL-XL doxycycline inducible transduction and establishment of iPS Cell lines that were then able to efficiently form definitive endoderm. B: Immunofluorescence and bright-field images showing induction of definitive endoderm from iPS cells after 6 days using protocol provided in FIG. 1D, with and without doxycycline, in iPSC lines with established doxycycline-inducible C-MYC, BCL-XL or control (no viral transduction) in cell line AS7017-5 (scale bar=100 μM), (n=2). C: Bright-field images from three inefficient iPSC lines induced with or without the addition of Doxycycline (no additional transgenes), scale bar=100 μM, (n=3); D: Schematic for optimal induction of most iPSC lines into definitive endoderm (7 days) and then to iPSC-derived hepatocytes. Represents Mattis Protocol 1 (MP1).

FIG. 2. Doxycycline Enhanced Endoderm. A: Immunofluorescence and bright-field images showing induction of iPSC line 1023-5 to definitive endoderm (DE) with Hoechst 33342 nuclear staining in blue, and antibody staining in green for OCT3/4, FOXA, HNF1p and CXCR4 with AlexaFluor 488 secondary (green) antibody. Bright-field microscopy shows complete monolayer. (scale bar=200 μM); B: Compound Screen showing other antibiotics and other compounds affecting the mitochondria were compared to doxycycline. While most antibiotics failed to have the same affect, surprisingly Gentamycin was shown to have a strong increasing dose response. In addition KB-R7943 and Ruthenium Red also showed responses at specific doses in one cell line (AG02261) but not another cell line (AS7192). The number of cells is shown measured seven days after induction compared to the plating density at day 0 (n=2); C: Comparison of Tetracycline Derivatives in iPSC lines AG02261. Doxycycline was compared to tetracycline derivatives in its ability to rescue endoderm induction. Minocycline, and Methacycline showed similar ability to rescue endoderm at the 1 μM concentration with mostly toxicity at the 10 μM concentration. Demeclocycline showed a similar trend. The number of cells is shown measured seven days after induction compared to the plating density at day 0. (n=2) Significant figures denoted by* indicates a p value of <0.05.

FIG. 3. Doxycycline Reduces Apoptosis and Increases Functional Mitochondrial Mass. A: Immunofluorescence of iPSC line 1023-5 on day 2 of induction with staining for Hoechst 33342, Ki-67, Cleaved Caspase 3 (CC3), and combined showed greatly decreased CC3 staining in the presence of doxycycline. At this early time point, (Day 2) there was a slight significant decrease in Ki-67. B: Mitotracker Orange staining of iPSCs with no induction and with induction showed that addition of Doxycycline increased mitochondrial activity compared to vehicle alone in three separate iPSC lines (AS7192-7, 1023-5, and AS7017-2). Significant figures denoted by*is p value less than 0.05 and**is p value less than 0.01

FIG. 4. Phosphorylated AKT Ratio Determines Definitive Endoderm Induction. A: RNA Sequencing of starting iPSCs, definitive endoderm, and final iPSC-Hepatocytes cluster appropriately after unbiased hierarchical clustering analysis, with and without doxycycline, enhanced differentiation during the endoderm phase only. B: Doxycycline-enhanced definitive endoderm induction was inhibited by Ly294002 in a dose-dependent manner (iPS line AS7192-7). C: Ly294002 inhibited P-AKT levels are not reversible by increasing doses of Doxycycline (iPS lines AG06103A-3 and AS7017-3). D: Pre-existing baseline ratio levels of pAKT/AKT in individual iPSC lines correlated with the ability to induce these lines to definitive endoderm. Significant figures denoted by*is p value less than 0.05 and**is p value less than 0.01.

FIG. 5. Optimization of Doxycycline and Difficult cases of Induction. A: Percentage well saturation during induction of cell lines 1 and 2 with vehicle (blue line), Y-27632 (red line), Doxycycline 1 μg/mL (green line), or Doxycycline plus Y-27632. B: Experiment with regents excluded in an attempt to minimize components within induction media. Our conclusion is that all reagents are required (CHIR=CHIR 99021). Significant figures denoted by*is p value less than 0.05 and**is p value less than 0.001. Error bars in A represent 95% range confidence interval.

FIG. 6. The standard protocol. A: The standard protocol disclosed herein for induction to endoderm and iPSC-derived hepatocytes (CHIR=CHIR 99021). B: Induction of iPSC with doxycycline-inducible cMyc still produced endoderm with markers of endoderm including A1AT, AFP, Albumin, HNF4A and FOXA2.

FIG. 7. Induction with Doxycycline and Dose Response. A: Comparison of control versus 1 μM Doxycycline (+Dox) induction over six different cell lines showed improved confluence and survival of cells. B: Titration of Doxycycline showed that there was an optimal level of doxycycline and a toxic level. Error bars represent 95% range confidence interval.

FIG. 8. Doxycycline Reduces Cleaved Caspase 3 Levels. A: Immunofluorescence of iPS cell line AS7192-9 two days after induction with staining for Hoechst 33342, Ki-67, Cleaved Caspase 3 (CC3), and combined showed greatly decreased cleaved caspase 3 in the presence of doxycycline. At this time point, there was a slight decrease in Ki-67. Significant figures denoted by*indicates a p value of <0.05.

FIG. 9. Comparison of Tetracycline Derivatives and Other Compounds in additional cell line. A: Induction of iPSC lines AS7192 was performed. Doxycycline was compared to tetracycline derivatives for the ability to rescue endoderm induction. Here Minocycline and Methacycline and to some extent Demeclocycline showed similar abilities to rescue endoderm at the 1 μM concentration, with both antibiotics showing no improvement at the 10 μM concentration. The number of cells was measured seven days after induction compared to the plating density at day 0. Significant figures denoted by*indicates a p value of <0.05.

FIG. 10. Doxycycline Increases Survival of cells during FACS and Effects of Plating Density on Confluencey after Endoderm Induction. A: Doxycycline increases the survival of cells during FACS sorting, especially for single cells B Titration of initial plating density of iPSCs versus doxycycline in 96-well plates showed that even low doses of docycycline rescued iPSC lines. The initial plating density is denoted by the marked chart on the right. Amount of doxycycline tested is shown in μg/mL on the X-axis. Significant figures denoted by*indicates a p value of <0.05.

### DETAILED DESCRIPTION

The disclosed methods have a variety of applications. For example, the methods disclosed herein provide for efficient differentiation of any PSC to endoderm for in vitro applications. In addition, the methods provide for efficient differentiation of any PSC to endoderm, and then to further differentiation to any endoderm derived tissue such as liver, beta-pancreatic cells, or lung alveolar cells for in vitro applications. The methods also provide for efficient differentiation of any PSC to endoderm and then further differentiation to any endoderm derived tissue such as liver, beta-pancreatic insulin secreting cells, or lung alveolar cells for in vivo transplantation into humans for whole organ or partial cellular replacement. The methods also provide for efficient differentiation of any PSC to endoderm that can be perpetuated or expanded indefinitely. Differentiation of PSC to endoderm using the MP1 with doxycycline method allows development of high-throughput screening using PSCs. For example PSCs are differentiated to endoderm or PSC to mature cells such as hepatocytes, beta-pancreatic cells or lung alveolar cells via the endoderm state in, e.g., 96-well or higher formats that are amenable to efficient, high-throughput screening of small molecules for a desired phenotypic effect. Also provided are uses of the new pluripotent cell lines for differentiation to endoderm and downstream cell lines.

Most PSCs differentiated currently fail to differentiate into endodermal cells. This technology overcomes this issue and allows for efficient endoderm induction. Moreover, PSCs that successfully differentiate are often inefficient producers of endoderm, yielding only small numbers of cells. The technology disclosed herein overcomes this problem and allows complete sheets of cells to be differentiated and/or allows cells to be effectively grown in 3 dimensions, including 3-dimensional balls or clusters of cells. The simple addition of the small molecule doxycycline improves PSC-to-endoderm differentiation to a degree not seen before. Addition of doxycycline inhibits cells death and drives the proliferation of cells. Differentiation of PSC to endoderm using the MP1 with doxycycline method provides for high-throughput or high-content screening using PSCs. Further, PSCs can be plated into 96-well or higher density plates, and differentiated to endoderm or other cells types such as hepatocytes, cholangiocytes, beta-pancreatic cells, or lung alveolar cells to provide the materials for high-throughput or high-content screens. The differentiated cells from different genetic backgrounds can then be tested for compounds, for genetic screens, or for toxicity or enhancement from chemicals or small molecules. Additionally the disclosed methods can be used to improve differentiation to terminal mature cells by allowing the testing of compounds on complete monolayers or on 3-dimensional clusters of cells. The efficient production of endoderm-derived tissues also allows for the individualized screening of tissues from individuals for phenotypic reactions to any compounds, including medications. It allows for screening of increased cellular death, metabolic changes, stress responses, increased autophagy or dysplastic changes that can progress to cancer.

The disclosed method was developed using an iterative experimental approach. Initially, the method included the use of either the cMyc gene or the gene BCL-XL under the control of doxycycline. The results, however, showed similar cellular proliferation profiles leading to the possibility that a common mechanism was involved. Further development led to data that showed, with appropriate controls, that doxycycline by itself induced cells to effectively grow into endoderm when added to base protocol without any exogenous genes. Neither cMyc nor BCL-XL were required for the proliferation and rescued endoderm phenotype. Doxycycline was sufficient to rescue the cell lines. It was then discovered that adding Y27632 to the protocol including doxycycline led to the rescue of additional stem cell lines during induction to endoderm. By itself, addition of Y-27632 to the protocol without doxycycline does not produce the same robust induction.

In somewhat greater detail, iPSC-Hepatocytes (i.e., iPSC-Heps) have been shown to effectively model single-nucleotide polymorphism (SNP) based inter-human metabolic differences including cytochrome P450 CYP2D6 type I and type II metabolic differences (1). More importantly, unlike hepatoma tumor lines, iPSC-hepatocytes function as post-mitotically differentiated cells without significant proliferation, but with the added benefit of true high-level hepatic functioning. Finally, unlike primary human hepatocytes, iPSCs can be readily expanded to differentiate into any number of hepatocytes and can genetically modify them in forward genetic screening. Thus iPSC-Heps have clear potential to be the most useful individual human-specific cell line to recapitulate not only our variable differences in SNP gene expression but also as a potential way to model our individual increased or decreased susceptibility to disease. Given its potential, the Achilles heel of iPSC-Heps has been the tremendous variability, expense, and inconsistency in differentiating these cells into hepatocytes. While embryonic stem cell lines show greater stability in differentiation, different iPS cells lines, derived even from the same patient, often fail to consistently differentiate into endoderm and often undergo massive apoptosis. IPSC-hepatocyte lines that make it past the endoderm stage, however, go on to successfully produce hepatocytes.

In the published literature and in practice, research groups often employ only several subclones of pluripotent cells for all of their studies, because they lack efficient induction of endoderm. In order to usefully characterize patient-derived iPSC lines from multiple patients using high-content screens, it is beneficial to consistently make true endoderm in complete sheets of monolayers. Furthermore, if these cells are to be used in more complex assays, such as Ussing chambers, then induction to complete monolayers is essential. We therefore explored the ability to achieve more efficient induction of endoderm.

Differentiation of stem cells into mature cell lines often progresses through a step-wise natural progression similar to embryogenesis. This differentiation progresses through the three germ layers, ectoderm, endoderm and mesoderm. For unknown reasons, production of endoderm appears to be particularly challenging in vitro and perhaps this is why spontaneous mature teratomas in vivo often contain more ectodermal and mesendodermal tissues than endoderm (2). Yet if the promise of iPS cells is to produce mature cell lines from any patient or healthy individual, this issue must be overcome. Induction of human iPS cells to endoderm via high-dose Activin A often produces significant apoptotic cell death. High-dose Activin A induces the noggin pathway, driving multiple pathways important in differentiation, including SMAD 2/3/4, and inhibition of P13 Kinase. Unfortunately, there are also multiple downstream pathways driving cellular apoptosis, including SHIP-1, DAP-Kinase, and TIEG (downstream of SMAD signaling), and inhibition of AKT pathways (via inhibition of PI3Kinase) (3-5). Because of these apoptotic pathways and significant loss of cells, successful induction is dependent on the health of the cell lines, the plating density, the media balance, and the surface environment, making differentiation to endoderm incredibly tricky and difficult. Even under optimal conditions, most iPSC lines undergo total apoptosis at the endoderm induction stage within the first two days. In fact, multiple investigators have categorized cell lines as either good or incompetent to form endoderm and its downstream products, like hepatocytes. We reasoned that the problem was a technical hurdle due to inappropriate Activin A activation of apoptotic pathways via SMAD signaling, and that there must be an efficient way to circumvent apoptosis and induce any cell line to form endoderm.

We therefore set out to test endoderm induction via genetic methods and we discovered that the addition of doxycycline to the differentiation protocol disclosed herein significantly enhanced endoderm and allowed differentiation of most iPS cell lines to form complete monolayers compatible with screening or transplant. We therefore fully explored the use of doxycycline to induce endoderm and finally differentiate these cells to iPSC-derived hepatocytes. The data disclosed herein show that doxycycline significantly improves survival and, thus, differentiation of iPSCs from multiple donors and different reprogramming conditions into sheets of endoderm cells with high efficiency. The results establish that there were only minimal changes in gene expression with the final differentiation, yielding nearly identical hepatocytes compared to standard published protocols. Characterization of the process disclosed herein identified apoptosis as one significant barrier that doxycycline was inhibiting, in addition to stimulation of phosphorylation of AKT, driving proliferation. Significant changes to mitochondrial metabolism with inhibition of oxidative phosphorylation were also shown, in contrast to prior studies. In cell lines that were still difficult to induce, the addition of Y-27632 (rho kinase inhibitor) in addition to doxycycline promoted the efficient and complete differentiation of these cell lines, allowing for downstream characterization. The disclosed methods efficiently differentiated greater than 62 of out 67 iPSC-derived hepatocytes to complete monolayers. In addition, the use of doxycycline allowed for the effective differentiation of cells in a 96-well format, efficiently making them amenable to screening and assays. Disclosed herein are data showing that doxycycline is an effective and compatible compound, with or without rock inhibitor, for the induction of iPS cells to endoderm. This has implications for cellular therapy, but also highlights the importance of understanding the effects of doxycycline on cellular metabolism and alternative pathways when used in models of gene regulation.

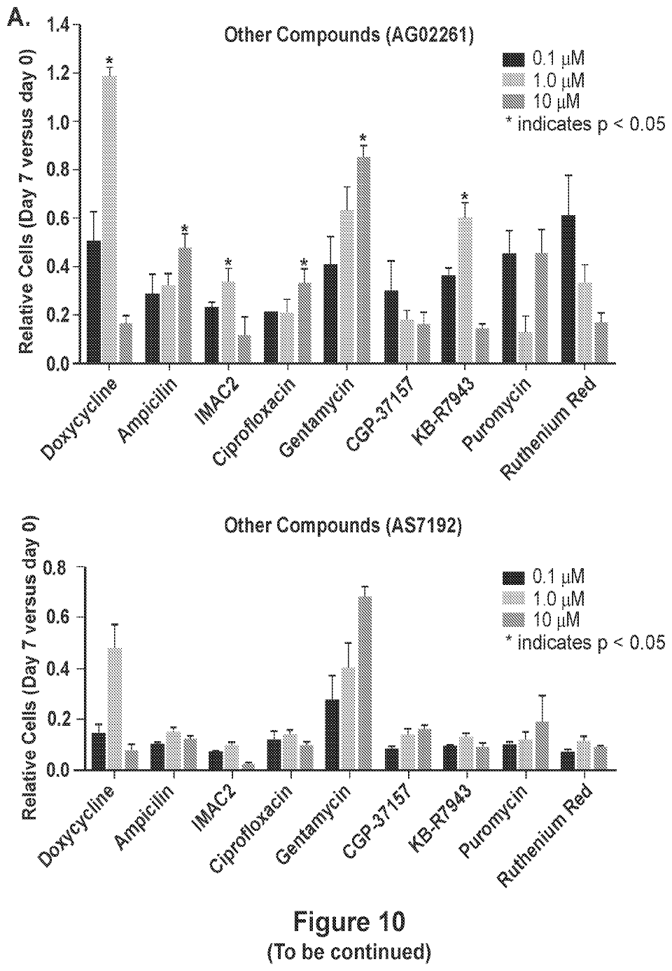

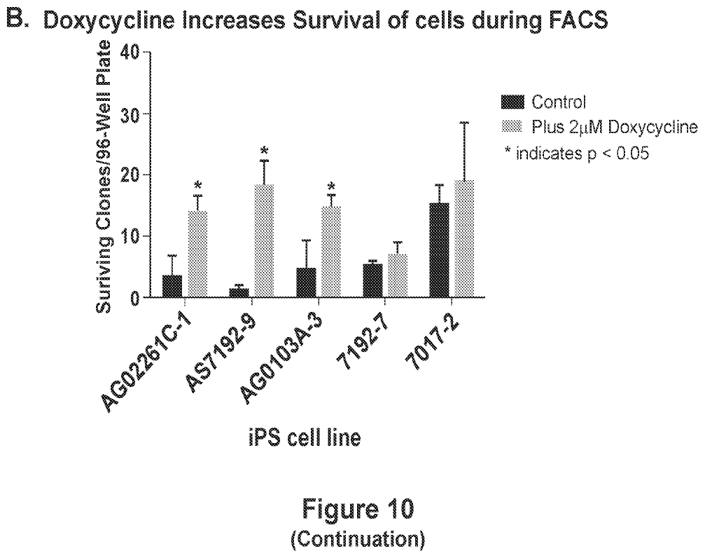

The experimental data disclosed in the following Examples established that doxycycline, perhaps via its many off-target binding sites, performs a dual role in endoderm induction. During the early first phase, it inhibited apoptosis, while during the late phase it promoted proliferation mediated via AKT phosphorylation. The use of the protocol disclosed herein has opened an entire differentiation pipeline that allows for investigations of aspects of cellular differentiation that had been blocked by the inefficiency of iPSC-derived hepatocyte endoderm induction. With the addition of doxycycline, the true power of in vitro differentiation of iPSC hepatocytes is now possible from any patient and almost any line making phenotype analysis possible and the comparison of patients more realistic in parallel experiments. In addition and consistent with doxycycline's known ability to facilitate iPSC cell growth (39), addition of doxycycline with Y-27632 improved iPSC survival during single-cell FACS sorting (FIG. 10). It is expected that the methods disclosed herein will lead to the complete maturation of iPSC hepatocytes into fully mature hepatocytes from each of the zonal areas of the liver lobe.

### EXAMPLES

### Example 1

### Materials and Methods

### Cell Culture of iPSCs

The media conditions to maintain human iPSCs have all been previously described (40). For passaging the cells Accutase (StemCell Technologies, Vancouver, BC) was used for 5 minutes at 37° C. and then stopped with iPSC media containing 10 μM Y-27632 (Selleckchem, Houston, Tex.) (41). The dissociated IPSCs were seeded on new plates with media containing Y-27632, and after 24 hours replaced with media alone. iPSC media was changed daily.

### Lentiviral Constructs and Protocol

Lentiviral constructs CS-TRE-c-MYC-PRE-Ubc-tTA-12G-RESS-GFP (cMYC) and CS-TRE-c-MYC-PRE-Ubc-tTA-12G-RESS-GFP (BCL-XL) are known in the art and were transduced as previously reported (14).

### FACS Analysis of Cells

After lentiviral infection by the Doxycycline-inducible cMYC or Doxycycline-inducible (BCL-XL) vectors, cells were analyzed on a FACS Ariall for GFP positive cells. Individual clones were isolated by sorting single iPSCs to wells of a 96-well plate precoated with Matrigel with iPSC media containing 10 μM Y-27632 and 2 μM doxycycline. Clonal iPSC isolates were expanded by weekly passaging and standard iPSC culture as described herein.

### iHEPS Differentiation

Induction of Definitive Endoderm (days 1-7). iPSCs were plated on 6-, 12-, or 96-well Matrigel-coated plates at different densities for differentiation in iPSC media containing 10 μM Y-27632 and 2 μM doxycycline. For example, typical plating density in a single well of a 6-well plate was 500,000 cells or 20,000 cells per well for 96-well plates. The differentiation process consisted of one week of RPMI media supplemented with Gem21 NeuroPlex without insulin at 1× (supplied at 50×, Gemini Bio-Products West Sacramento, Calif., catalog #400-962, or supplemented with Gibco B27 without insulin at 1×), Glutagro at 1× (Corning, Manassas, Va., catalog #25-015-CI, non-essential amino acids at 1× (Gibco, catalog #11-140-050), sodium butyrate (0.5 mM, Sigma Aldrich, catalog #B5887) and recombinant human Activin A (100 ng/mL, Peprotech, Rocky Hill, N.J., catalog #120-14E) for 7 days. On day 1, 2% KnockOut Serum Replacement (KSR; Gibco Catalog #10828028), CHIR-99021 (10 ng/mL, Selleckchem catalog #S2924), PI-103 (10 ng/mL,Selleckchem catalog #S1038), recombinant human BMP4 (10 ng/mL, Peprotech catalog #120-05ET) and FGF2 (20 ng/mL, Peprotech catalog #100-18B) were added to the media. On day 2, 1% KSR, PI-103 (10 ng/mL) and BMP4 (10 ng/mL) and FGF2 (20 ng/mL) and on day 3, 0.2% KSR and PI-103 (10 ng/mL) were added to the media. Doxycycline hyclate (Acros Organics catalog #446061000) was added throughout definitive endoderm induction starting on day 0 (passage day) at various concentrations, as described herein, with a standard concentration being 2 μM. These steps can be performed at normal room oxygen at about 20% or at low oxygen between 4 and 6%.

Differentiation of Definitive Endoderm to hepatocytes (days 8-23). iPSC-derived human endoderm prepared as described herein was cultured continuously on Matrigel or split 1:2 onto Matrigel plates for hepatic differentiation. Definitive endoderm (DE) was cultured in Iscove's modified Dulbecco's medium (Gibco catalog #supplemented with Gem21 NeuroPlex without insulin (Gemini Bio-Products catalog #400-962), Glutagro, non-essential amino acids (1×, Gibco catalog #11-140-050), 0.3 mM monothioglycerol, 5 μg/mL human insulin (Sigma), and 100 nM dexamethasone. To induce hepatoblasts, cells were treated with FGF2 (10 ng/mL) and BMP4 (20 ng/mL) for 5 days. During this phase, cells can be passaged using accutase and split at least 2 to 4× to increase yield and quality of final product again onto Matrigel with the addition of 10 μM Y-27632 and 2 μM doxycycline to increase cell survival during accutase treatment and splitting. This phase and the next were grown at low oxygen (4-6%) and 5% CO₂.

To further differentiate the cells towards hepatocytes, cells were treated with FGF2 (10 ng/mL), BMP4 (20 ng/mL), and recombinant human HGF (20 ng/mL, Peprotech catalog #100-39) for 5 days followed by changing the culture conditions to Lonza Hepatocyte Culture Media BulletKit (HCM catalog #CC-3198) with HGF (20 ng/mL) and recombinant human Oncostatin M (20 ng/mL, Peprotech catalog #300-10) for 5 additional days. In the BulletKit, use of EGF was excluded. This last BulleKit phase was grown at either low oxygen (4-6%) and 5% CO₂ or at 20% oxygen and 5% CO₂.

Immunohistochemistry and Cell staining. iPSCs, endodermal cells and iHeps were fixed using 4% paraformaldehyde for 15 minutes, permeabilized using 0.1% Triton X-100 for 10 minutes followed by 3× PBS (phosphate buffered saline, Corning Life Sciences) rinses and blocking with 1.5% bovine serum albumin for 1 hour. Samples were incubated with primary antibody overnight. A list of antigens specifically bound by all antibodies with dilutions in blocking solution indicated parenthetically: OCT3/4 (Santa Cruz, #sc-9081, 1:500), SOX2 (Proteintech, #11064-1-AP, 1:1000), NANOG (Proteintech, #14295-1-AP, 1:1000), A1AT (R&D Systems, catalog #AF1268, 1:500), C8/18 (Cell Signaling, catalog #4546 Ms, 1:200), SOX17 (Santa Cruz Biotechnology, catalog #sc376126, 1:1000), FOXA2 (Santa Cruz Biotechnology, catalog #5c6554, 1:250), HNF1p (Santa Cruz Biotechnology, catalog #sc130407, 1:500), CXCR4 (Santa Cruz Biotechnology, sc53534, 1:250)−AKT (Cell Signaling, catalog #40D4, 1:500), P-AKT (Cell Signaling, catalog #S473, 1:500), TRA 1-60 (Proteintech, catalog #18150-1-AP, 1:500), HNF4α (Proteintech, catalog #26245-1-AP, 1:500, Human Albumin (Bethyl, catalog #A80-129A, 1:500, AFP (Thermo Scientific, catalog #RB-365-A1, 1:500), ki-67 (8D5) (Cell Signaling, catalog #9449, 1:800), and Cleaved Caspase-3 (Cell Signaling, catalog #9661, 1:400). The cells were rinsed extensively with PBS and incubated for 60 minutes with secondary antibody at 1:1000 dilution in blocking solution. The secondary antibodies used were: Alexa Fluor 555 donkey anti-mouse IgG (H+L), Alexa Fluor 488 goat anti-rabbit (H+L) (Invitrogen, Carlsbad, Calif.). After washing 3 times with PBS, DAPI (4′,6-diamidino-2-phenylindole, MP Biomedicals, Solon, Ohio) was added at 300 nM for 15 minutes. After 3× PBS washes, the cells were imaged using a BioTek Cytation 5 imager (Winooski, Vt.). MitoTracker Orange CMTMRos staining was performed as follows: iPSCs were plated, induced 24 hours with or without 1 mM doxycycline, and 24 hours later, Mitotracker Orange dye was added for 45 minutes in PBS buffer at 37° C. for 30 minutes, and then rinsed in PBS. Cells were then fixed with 4% PFA and Hoechst 33342, rinsed in PBS, and imaged with Cytation5 imager, BioTek Instruments.

RNA isolation, Real-Time PCR analysis, and RNA Sequencing. Total RNA was isolated by Qiagen RNeasy mini kit or Zymo Direct-zol RNA kits. Complementary DNA (cDNA) was synthesized by qScript cDNA SuperMix (Quantabio). Quantitative Real-Time PCR was performed using FastStart Universal SYBR Green, Roche Diagnostics (Indianapolis, IN). RNA Sequencing was performed by BGI, Boston, Mass. Poly-A RNA was selected using oligo-dT magnetic beads followed by N6 random priming. On average, 27,642,407 raw reads were obtained and mapped. Raw reads were subjected to quality control. Clean reads were analyzed by Gene Ontology analysis, KEGG pathway enrichment, and cluster analysis.

Moon Bio Antibody Array. To analyze protein levels and specific phosphorylation, the Phospho Explorer Antibody Array was performed. iPSC line 7192 was induced with or without 1 doxycycline for 3 days. Extracts were made using Complete Lysis-M EDTA-Free Lysis buffer (Roche). Antibody Array was performed, scanned, and analyzed by Full Moon Biosystems, Sunnyvale, Calif.

Western blot analysis. Western blots were prepared and used as previously described (42).

Statistical Analysis. Experiments were run in at least triplicate for each condition. Statistical significance was determined using Student t test (GraphPad Prism, La Jolla, Calif.).

### Example 2

Induction of Multiple iPS Cell lines from Different Patients using Defined Media

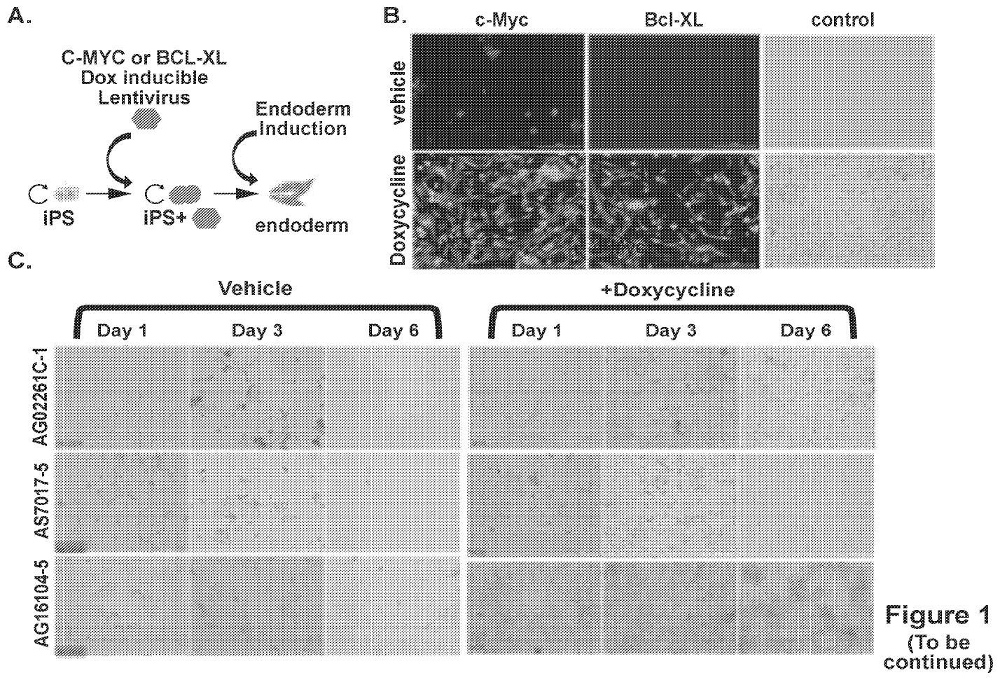

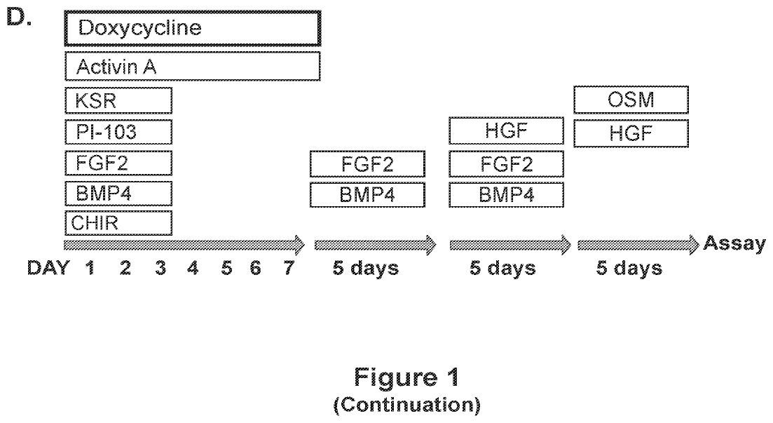

An interest in characterizing multiple different iPS patient cell lines led to testing that revealed significant differentiation variability even among popular published protocols (6-8). In order to better optimize the differentiation methods, multiple different batches of Matrigel were tested using poorly differentiating cell lines to screen for Matrigel lots that would best support a basic differentiation protocol based on published protocols (6, 7). We selected one Matrigel lot for all further experiments based on its ability to better support our weakest cell lines; however, even with this Matrigel, we did not see efficient differentiation and we often saw significant cellular death (FIG. 1C). Because we were interested in characterizing many different lines from different patient iPSC-Hepatocytes, we next set out to test combinations of different protocols to see if we could achieve the best possible differentiation across multiple different cell lines. Addition of CHIR99021 (Wnt agonist) in the first 24 hours improved the number of cells in multiple different cell lines (10). Addition of FGF2 and BMP2 for the first 48 hours sometimes helped endoderm induction and other times was not obviously helpful (10). We chose to retain these factors in the methods disclosed herein to achieve the best possible endoderm (11).

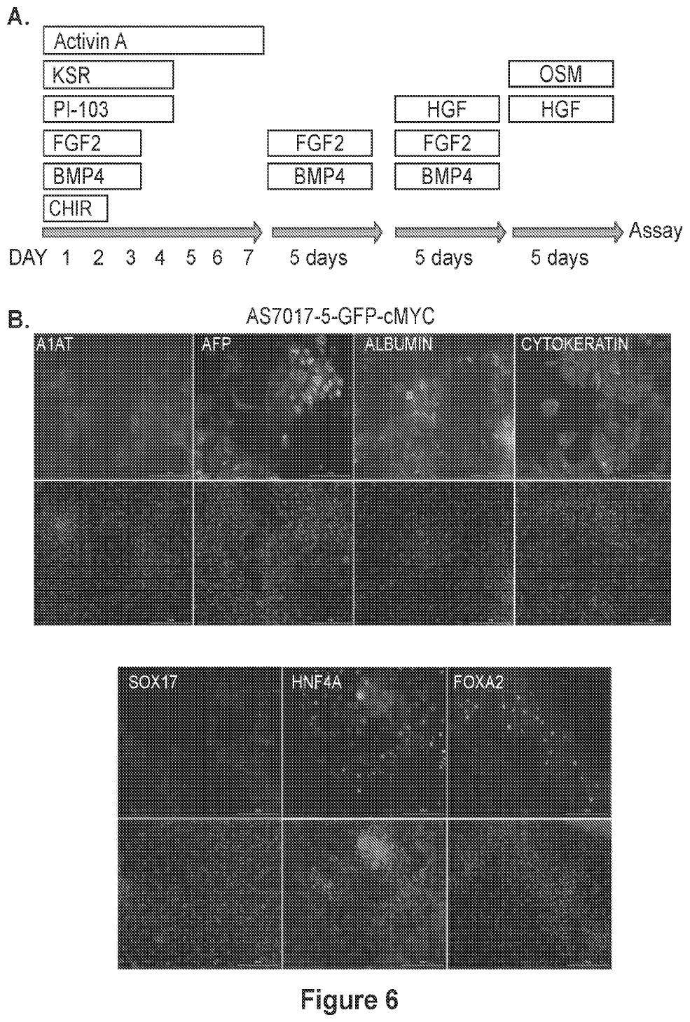

Based on our experience of using feeders with induction in serum-free media (7, 12), we kept serum out of many early experiments. In frustration with very sick cell lines, however, we finally added knockout serum replacement (KSR) and found that addition of small amounts of KSR in the first 72 hours improved the survival of some cell lines. In order to counteract the insulin present in KSR, we added the small molecule PI-103 (P13 kinase inhibitor) (12, 13). This combined protocol (FIG. 6A), worked fairly well for about one fourth of our cell lines but still showed stochastic cell death, inconsistency, and did not allow us to differentiate numerous important patient-derived iPSCs.

### Example 3

Induction of BCL-XL or cMYC by Doxycycline inducible Lentivirus increases Endoderm Proliferation

We considered the possibility that if we could artificially drive proliferation while inhibiting apoptosis, then perhaps we could at least short-circuit cell death and drive endoderm induction in most of our cell lines. We chose poorly differentiating iPS cell lines (AG02261c-1, 7017-6, AG16104-5; FIG. 1), and infected them with green fluorescent protein(GFP)-tagged-lentivirus expressing Dox-On BCL-XL or Dox-On cMYC (FIG. 1A and 1B). Because these lentiviral integrated cells contained the green fluorescent protein (GFP), we used fluorescence-activated cell sorting (FACS) to isolate clonal GFP-positive iPS subclones and expanded pure populations of these iPSCs containing integrated BCL-XL or cMYC (FIG. 6; FACS of GFP+cells). Next, we tested endoderm induction, by providing doxycycline to these cell lines and inducing BCL-XL or cMYC (FIG. 1 B). Using this system, three poorly differentiating cell lines were immediately differentiated when either cMYC or BCL-XL were induced by doxycycline (FIG. 1B and FIG. 6). BCL-XL or cMYC induction were confirmed by quantitative PCR. The rescued differentiation of iPSC lines that normally underwent apoptosis was impressive, and even more importantly the differentiated cells were forming endoderm, as confirmed by immunohistochemistry (FIG. 6). These findings were unexpected because others had previously reported that deletion of cMYC was necessary for spontaneous primitive endoderm differentiation from iPS cells (15). The results disclosed herein showed that activated cMYC expression is entirely compatible with endoderm induction.

### Example 4

### Doxycycline Rescues Endoderm Induction at different Cellular Densities

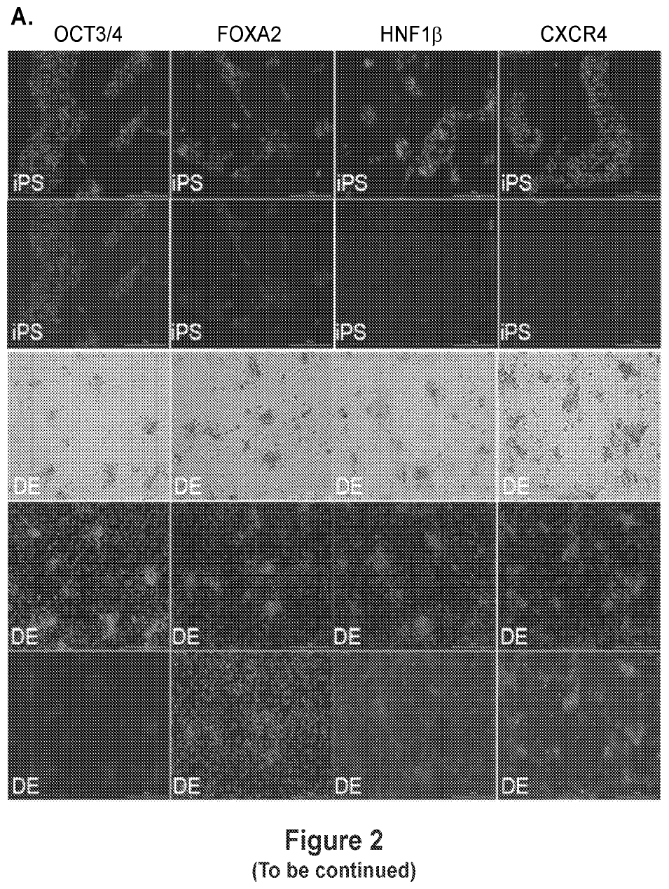

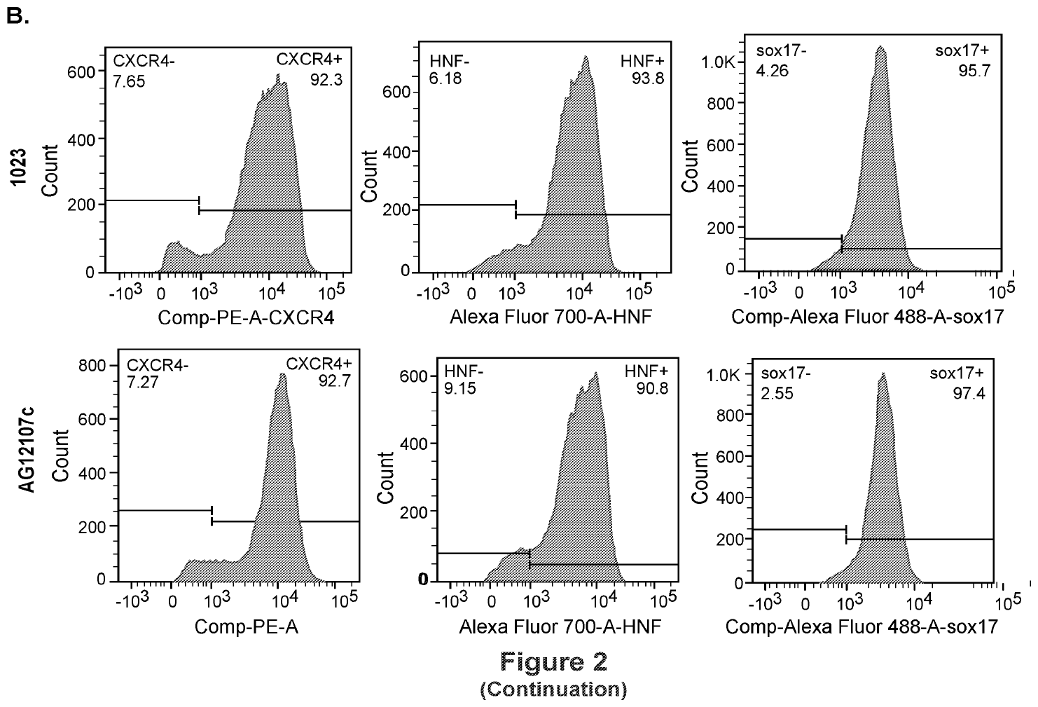

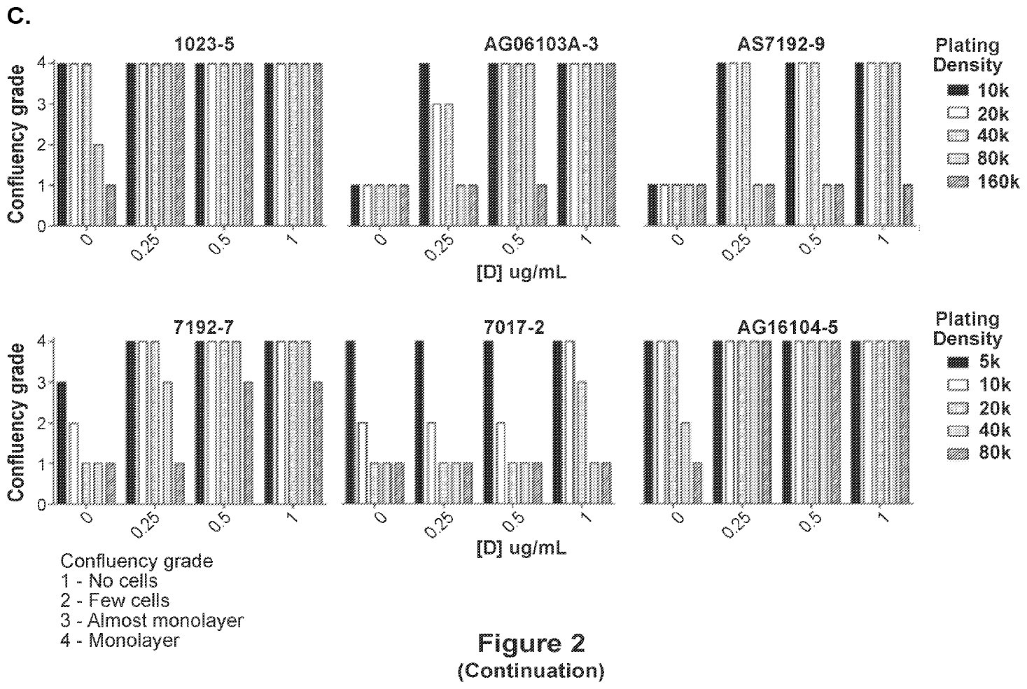

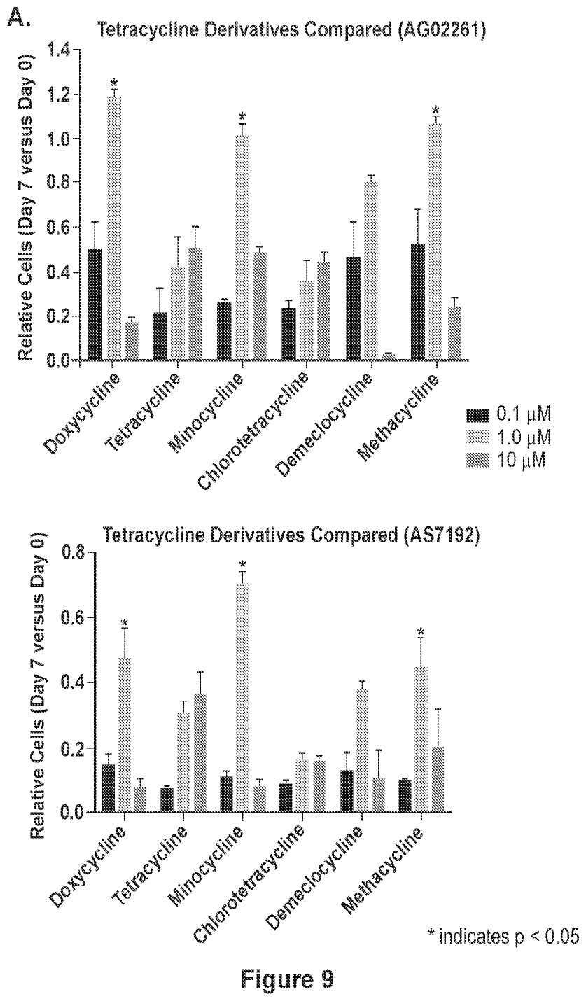

Our results were striking and somewhat unexpected, and because naïve iPS cell lines without the integrated lentivirus should be a great negative control, we performed this experiment (with and without doxycycline induction). Upon testing the inductions using lentiviral-free controls, we were surprised that doxycycline by itself rescued induction of iPSC-derived endoderm (FIG. 1 B). This result was confirmed using multiple different poorly differentiating cell lines (FIG. 1C). With this discovery, we abandoned the use of lentivirally transduced iPSCs and focused on characterizing the effects of doxycycline on iPSC-derived endoderm. To further analyze the ability of doxycycline to induce endoderm, we titrated iPSC seeding density versus doxycycline doses on six different lines with variable inducibility from completely uninducible to inducible without doxycycline (FIG. 10B). At even the lowest doses of doxycycline, there was an immediate improvement in all six lines tested. Most were able to induce to confluency and avoided cell death. We tested other antibiotic classes including ampicillin, ciprofloxacin, Gentamycin, and Puromycin. Compared to doxycycline, only Gentamicin showed a similar statistically significant ability to enhance survival of cells during induction to endoderm (FIGS. 2B and 9B). We also tested tetracycline derivatives for their abilities to rescue induction to endoderm, and compared to doxycycline, only minocycline, demeclocycline and methacycline showed similar trends in two different cell lines. The parent compound tetracycline and derivative chlorotetracycline did not appear to work (FIGS. 2C and 9A).

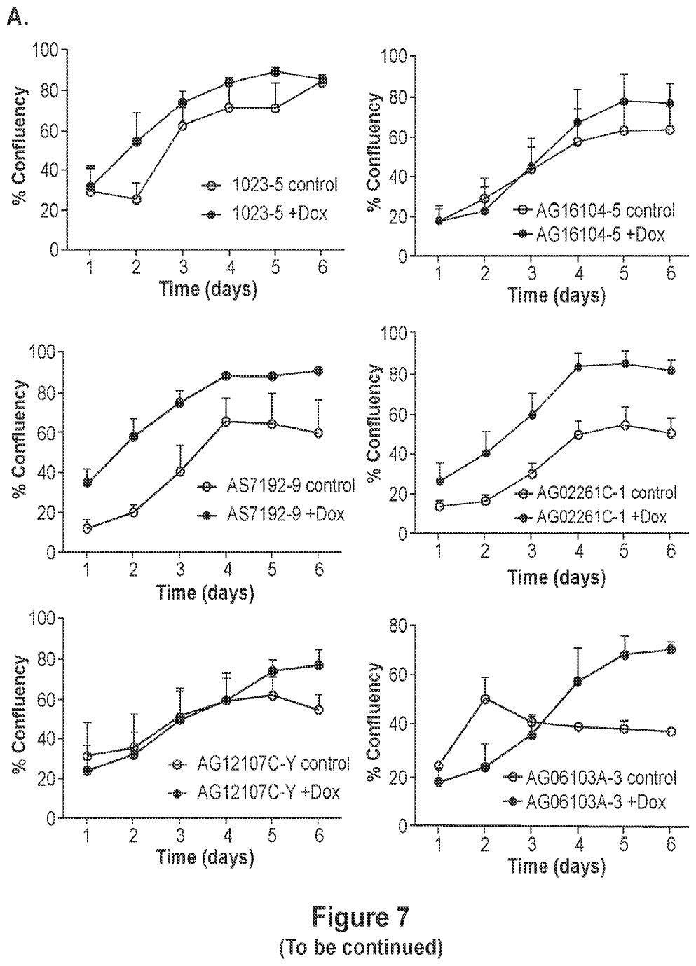

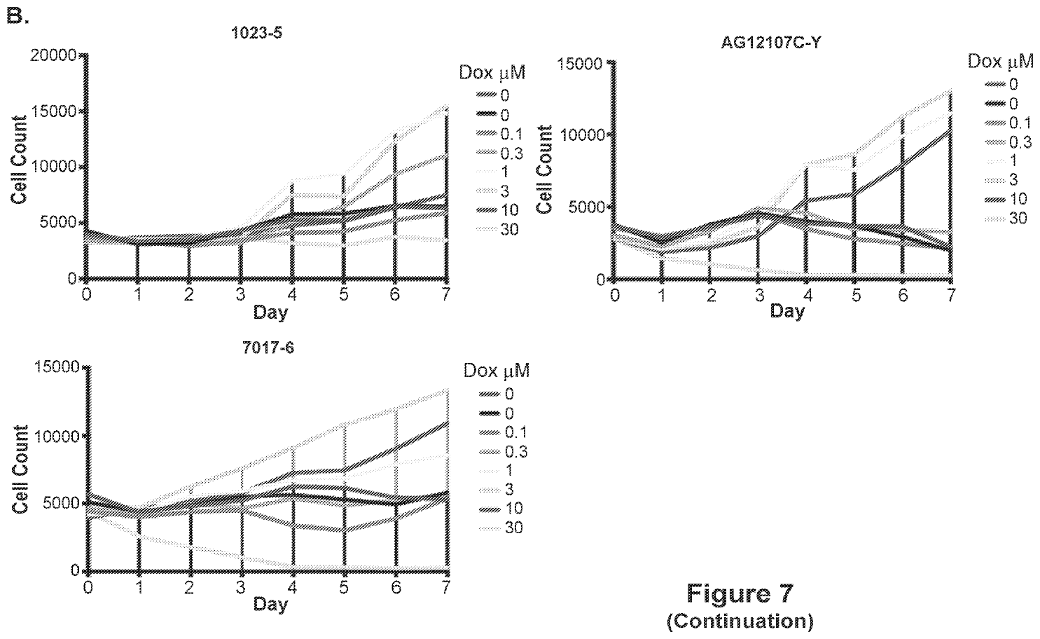

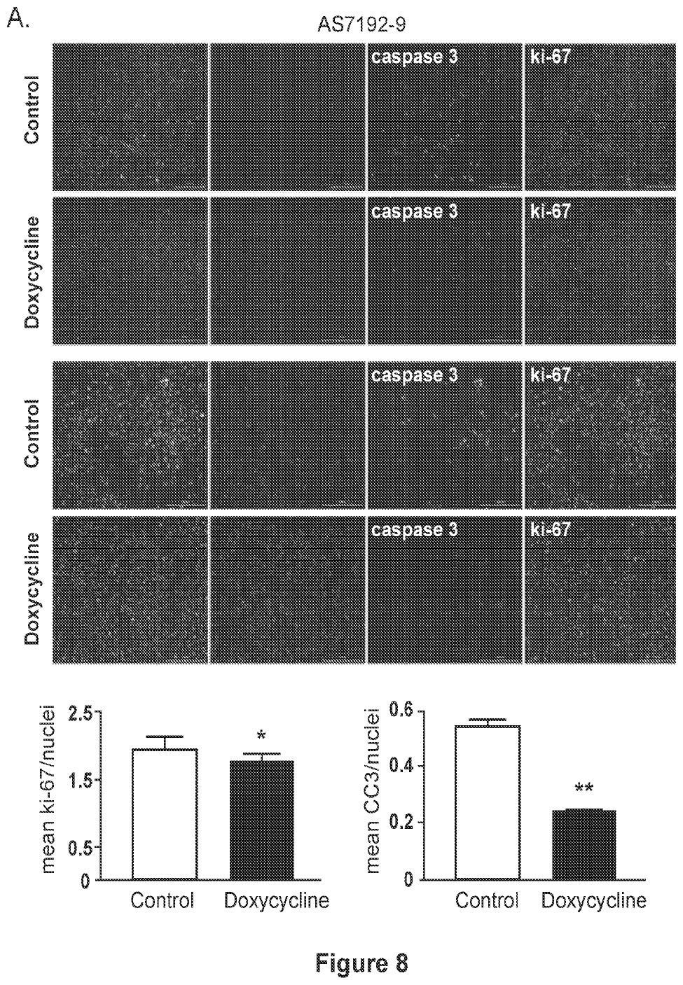

Given our serendipitous finding, we next tested the effects of doxycycline on iPSC growth and found that it was able to slightly improve daily growth kinetics in nearly all lines tested (FIG. 7A). We also did a complete dose-response curve on the growth of iPS cells in the presence of doxycycline up to 30 μg/mL, with toxicity observed beginning at the 10 μg/mL level (FIG. 7B). Beyond effects on iPS cells, we further confirmed the ability of doxycycline to support the induction of iPSCs to endoderm. For this we performed immunohistochemical staining for markers of endoderm, including CXCR4, SOX17, FOXA2, and HNF16 (FIG. 2A and 2B). Induction of endoderm showed loss of OCT 3-4 staining with induction of nuclear FOXA2, HNF16, and CXCR4 (FIG. 2A). SOX17 was transiently increased. The Mattis Lab One (ML1) protocol disclosed herein showed efficient induction of endoderm. More importantly, the ML1 protocol allowed induction of complete monolayers of endoderm from virtually any iPSC. Compared to many of the other protocols that we tested for poorly differentiating iPS cell lines(6, 8), we could actually differentiate most lines using the ML1 protocol, but only a fraction of them using the other protocols. To further understand if we could maximize cellular proliferation by a dose-dependent increase of doxycycline, we performed a titration curve for 6 different iPS cell lines, including ones that were resistant to differentiation by using a protocol disclosed herein that lacked doxycycline (FIG. 10B). The data show that addition of even small concentrations of doxycycline across a variety of plated cell densities was able to improve nearly all of the iPS cell lines tested for endoderm induction, and also facilitated formation of complete monolayers in many of the lines down to 96-well plate formats, something that was difficult to accomplish without using a protocol disclosed herein. Some cell lines required higher doses of doxycycline for maximum effect. The cells tolerated concentrations of doxycycline up to 10 μg/mL. At higher concentrations, the doxycycline showed toxicity with total cell death by the third day of induction at the highest concentrations (>10 μg/mL) (FIG. 7B). The maximum effect of doxycycline cellular survival in most lines was noted at concentration ranges between 0.1 μg/mL and 3 μg/mL, making the compound useful and nontoxic over its functional range.

### Example 5

Reduced levels of cleaved-caspase 3 cleavage

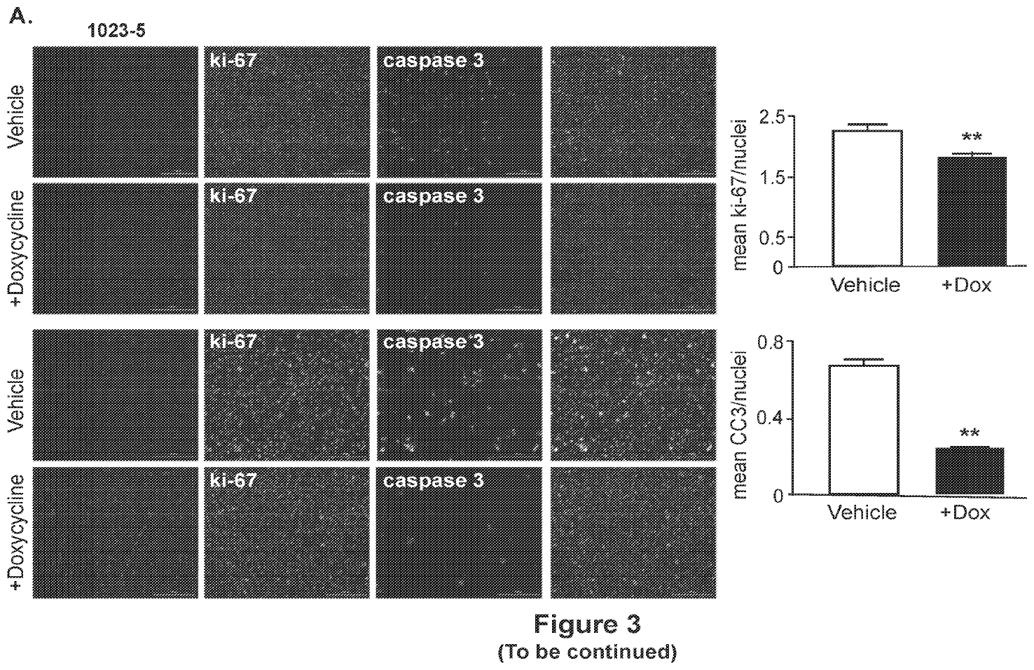

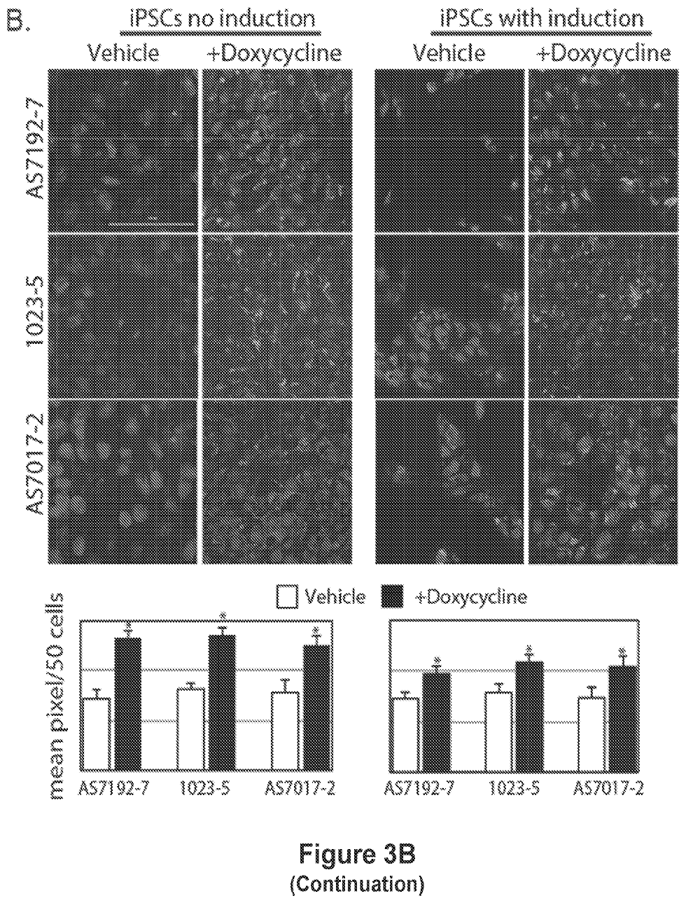

As a first step to understand the mechanism by which doxycycline was affecting induction of iPSCs into endoderm, we explored whether doxycycline was affecting proliferation versus cellular death. For this, we performed an induction for endoderm using protocol ML1 for 48 hours and then fixed and stained the cells for cleaved caspase 3 (CC3) and the proliferation marker Ki-67. After quantification, we noted both significant Ki-67 staining and cleaved caspase 3 (CC3) staining, as expected (FIG. 3A). In the presence of doxycycline, during this early induction timepoint, we noted a greater than 50% decrease in CC3 staining with only a very slight decrease in overall Ki-67 levels over 4 different cell-line experiments. This early reduction in apoptosis leads to sustained proliferation leading to successful induction.

### Example 6

### Interrogation of Mechanism Reveals Multiple Pathway Interactions

Given its function as an antibiotic, early studies of doxycycline have shown that it functions as a mitochondrial translational inhibitor (16). Subsequent studies have shown additional functions. For example, in tumor lines, doxycycline can alter numerous metabolic genes and furthermore may affect tumor and non-tumor cells differently, possibly because of metabolic differences (17, 18), with even long-term epigenetic modifications noted in T-cells after treatment (19). The precise mechanism underlying the ability of doxycycline to enhance the induction of endoderm, however, was not apparent. For example, there have been studies showing that maintenance of pluripotency is a metabolic process, and that during successful endoderm induction, there is a switch to oxidative phosphorylation (20, 21). Disclosed herein, however, is data showing that doxycycline was able to induce cells and to induce cMYC during transformation to endoderm, without negative effects on net endoderm or final iHep differentiation. Doxycycline is known to inhibit mitochondrial function and therefore reduce oxidative phosphorylation. Cliff et al. performed a similar experiment focused on metabolism during endoderm induction that measured cMYC levels during days 3 and 4 of endoderm induction but did not fully quantify the effects over multiple cell lines or to final differentiation towards iPSC-derived hepatocytes (22). Inhibition of mitochondrial metabolism and successful induction of endoderm is contrary to the notion that endoderm induction requires an oxidative metabolism state. The results disclosed herein show that this is indeed unnecessary for the production of SOX17/FOXA2-positive cells that can go on to form hepatocytes.

We expected that TGF-activated differentiation protocols involving Activin A would limit cell division because, by itself, Activin A shuts down cellular proliferation and under certain cellular scenarios activates the apoptotic machinery. Without wishing to be bound by theory, we reasoned that perhaps by inhibiting mitochondrial function, we were interfering with the ability of the mitochondrial apoptosis machinery to fully engage. To characterize mitochondria, we performed Mitotracker staining, with and without doxycycline, on iPS cells and during induction of endoderm (FIG. 3B). Treatment of cells with doxycycline showed increased MitoTracker Orange CMTMRos staining, consistent with an increase of functional mitochondrial mass, as oxidation is required for fluorescence. This experiment was repeated it three times with similar results. The results showed that doxycycline does not affect mitochondria in iPSCs and induced iPSCs in the same way that it does in cancer stem cells (17, 23, 24).

We further sought to delineate the pathways that were leading to increased apoptosis during the endoderm inductive phase. Induction of endoderm by high-dose activin A is thought to drive the nodal pathways by binding Activin A receptors 1 and 2, thus driving the phosphorylation of SMAD2 and 3 (25). The Phospho-SMAD2/3 complex recruits SMAD4 and enters the nucleus to drive transcription of multiple downstream targets (25). There are several published mechanisms for activated SMAD2/3/4 connecting to the apoptotic pathway. First, TGFβ is known to induce apoptosis through SMAD-mediated DAP-kinase expression (26). Second, TGFβ receptor (Activin A-activated) is also directly connected to a SMAD4 mitochondrial translocation and cyctochrome C oxidase subunit II interaction (27). These findings might explain the apoptotic propensity of iPSCs from certain cell lines during endoderm differentiation.

In order to find possible mechanisms involved, we performed RNA Sequencing from 5 different iPS cell lines induced to endoderm in pairs, with and without doxycycline. Upregulated and downregulated RNA pathways are shown in Table 1. We found 205 genes were significantly downregulated and 47 upregulated (adjusted p value of 0.05). Ingenuity Pathway Analysis (IPA) was performed, which revealed Cellular Growth and Proliferation as well as Cell Death and Survival as within top Molecular and Cellular Functions. However, these pathways appeared largely balanced, without clear targets that were overwhelmingly affected. Notable levels of Caspase 3 were minimally but significantly reduced. In addition, inhibition of matrix metalloproteases (MMPs), a known target of doxycycline, was a top canonical pathway. The connection between MMPs and apoptosis has been explored (28); however, only TIMP4 inhibition would be consistent with decreased apoptosis in this data set.

To further explore affected protein pathways including protein phosphorylation that would not be detected at the RNA level, we also performed a quantitative antibody array (Moon Bio, Sunnyvale, Calif.) on protein extracts from an induced iPSC-endoderm line, with and without doxycycline (Table 2). Increased phosphorylations were noted in in protein pathways driving proliferation and cell survival, including Elk-1 Ser383, ACK1 Try284, Dok-2 Tyr299, MEF2C Ser396, and HER2 Tyr1221/Tyr1222 (29-33). It was noted that phosphorylation of ACK1 Tyr284 has been shown to lead to increased AKT phosphorylation (29). With these activated pathways in mind, we revisited the RNA expression pathways and noted that the ERK pathway that induces apoptosis was also affected via downregulation of CAV1 (34) and that, furthermore, CAV1 can also interact with the TGF-beta receptor influencing SMA2 phosphorylation (35).

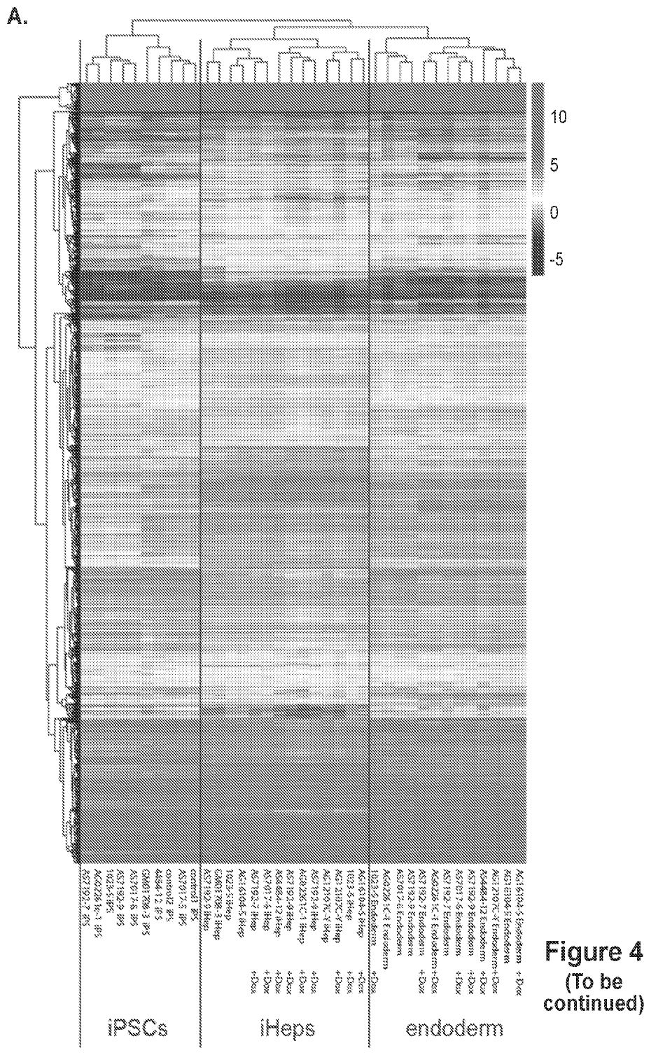

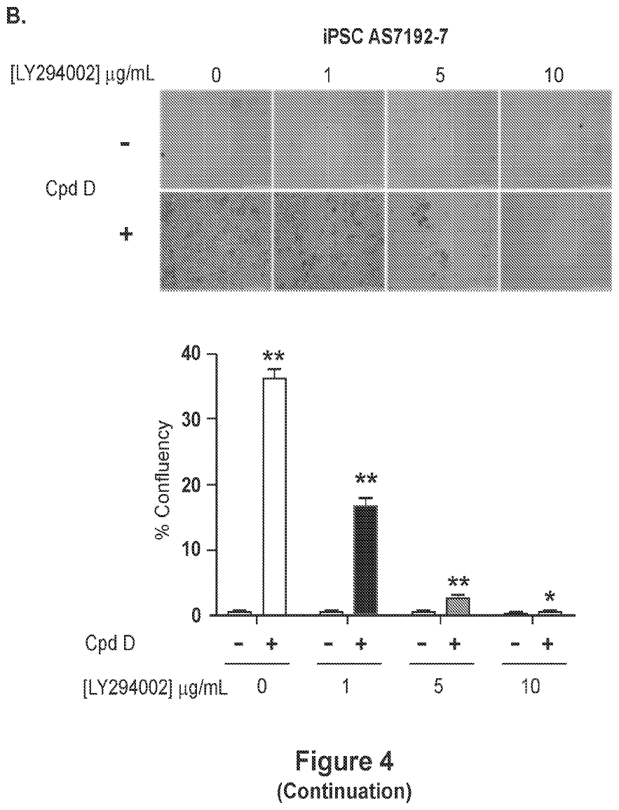

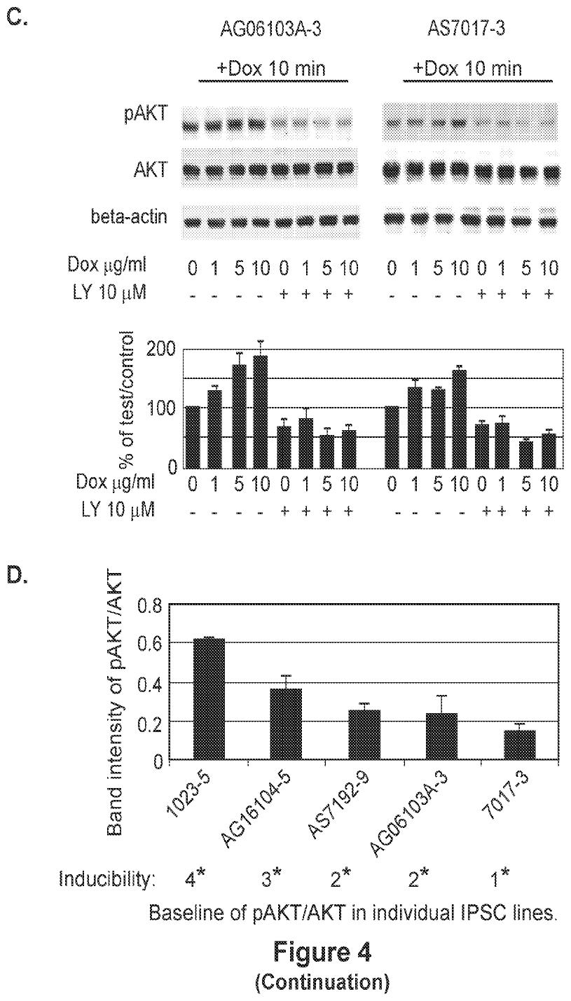

As a next step and to confirm that AKT indeed showed increased phosphorylation in the presence of doxycycline, we used four iPSCs and performed a doxycycline dose-response experiment (FIGS. 4B and 4C). This was further confirmed by Western blot analysis showing a correlation of increased doxycycline with AKT-phosphorylation, which was entirely reversible with LY294 dose-dependent inhibition.

The reason for different iPSCs showing different propensities for Activin A sensitivity to cell death was not apparent, however. AKT directly interacts with unphosphorylated SMAD3, through a mechanisms independent of AKT kinase activity. By sequestering SMAD3, the complex can default into apoptosis (36). Therefore, pre-existing levels of phosphorylated AKT could determine the efficiency of induction and this may hold the key balance. In order to characterize if such levels could be leading to the noted differences between cell lines, we characterized the ability of cells to differentiate into endoderm and avoid apoptosis as excellent (four stars), average (three stars), and poorly inducible cell lines (2 and 1 stars). We then compared preexisting levels of Phosph-AKT/AKT levels in these cell lines (FIG. 4D). Excellent inducers showed baseline P-AKT/AKT ratios of greater than 0.3 versus poor inducers.

### Example 7

### Enhanced Endoderm Induction does not Alter the Final Hepatocyte Maturity

An experiment was designed to confirm that use of doxycycline did not affect the final iPSC-derived hepatocytes. To characterize the effect of doxycycline on the final iPSC-derived hepatocytes, we performed RNA Sequencing was performed on 5 paired iPSCs fully differentiated to hepatocytes using the protocol disclosed herein. Principal component analysis showed that use of doxycycline did not alter the final product (FIG. 4A).

### Example 8

Addition of Y-27632 plus doxycycline allowed induction of severely induction resistant cell lines

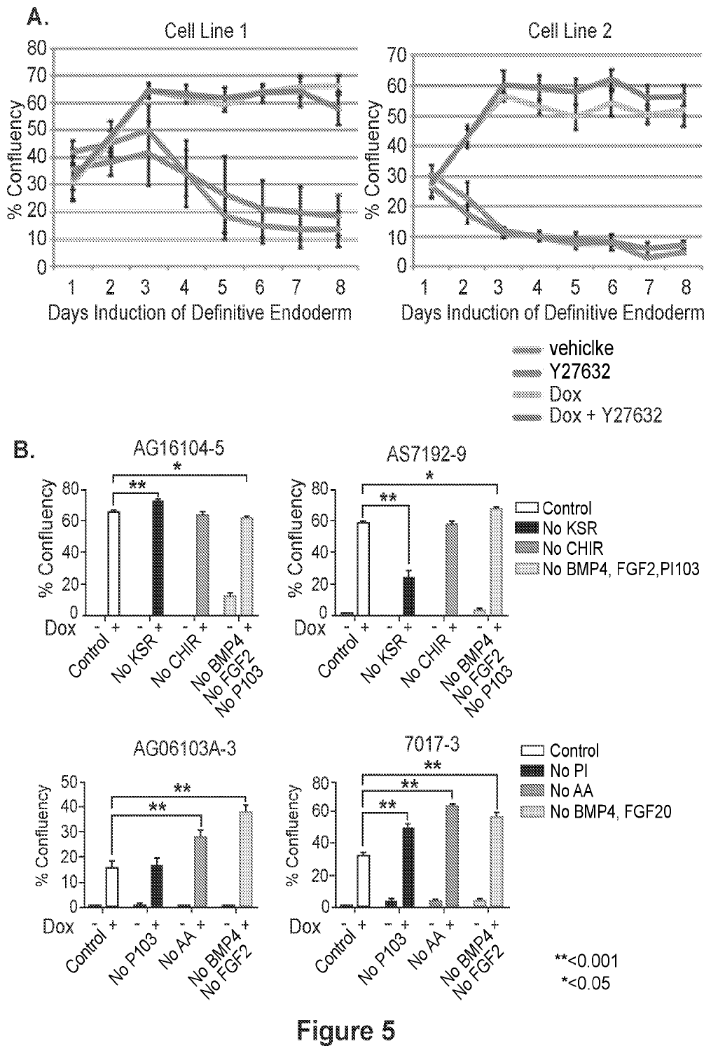

Based on our findings, experiments were performed in which a different, single component of the differentiation protocol disclosed herein was omitted. Using this approach, we were unable to exclude any component without loss of differentiation efficiency (FIG. 5B). We did note some rare cell lines that continued to undergo significant apoptosis. Realizing that apoptosis can be driven by additional pathways, such as the Rho kinase pathway (37), we tested the addition of Y-27632. By itself, Y-27632 was not able to rescue resistant lines during induction to iPSC-derived endoderm. Because doxycycline and Y-27632 are considered to work on different pathways, we tried adding doxycycline and Y-27632 together to poorly differentiating cell lines (FIG. 5A). Using this modified protocol, we were able to differentiate 70 iPSCs from 42 different patients to fully confluent wells for downstream analysis.

All patents and other publications identified are expressly incorporated herein by reference in their entirety or in relevant part as would be apparent to one of ordinary skill in the art from context, the incorporation effectively describing and disclosing, for example, the methodologies described in such publications that might be used in connection with information disclosed herein.

What is claimed is:

1. A method of inducing differentiation of a pluripotent stem cell (PSC) to a definitive endoderm cell comprising incubating the PSC in cell culture media supplemented with Activin A and an antibiotic, thereby producing a definitive endoderm cell.

2. The method of claim 1 wherein the PSC is an induced pluripotent stem cell (iPSC).

3. The method of claim 1 wherein the cell culture media comprises an inhibitor of caspase 3 cleavage.

4. The method of claim 3 wherein the inhibitor of caspase 3 cleavage is doxycycline.

5. The method of claim 1 further comprising contacting the PSC with a Matrigel-coated surface.

6. The method of claim 1 wherein the PSC is incubated in cell culture media for about 3 to 7 days.

7. The method of claim 6 wherein the PSC is incubated in cell culture media for 7 days.

8. The method of claim 1 wherein the cell culture media is RPMI media supplemented with Gem21 NeuroPlex without insulin at lx concentration, Glutagro at about 2 mM, MEM non-essential amino acids at 1× concentration, about 0.5 mM sodium butyrate, about 100 ng/mL Activin A, and 1-3 μM doxycycline.

9. The method of claim 1 further comprising adding about 2% KnockOut Serum Replacement (KSR), about 10 ng/mL CHIR-99021, about 10 ng/mL PI-103, about 10 ng/mL BMP4, and about 20 ng/mL FGF2 on day 1 of incubation.

10. The method of claim 1 further comprising adding about 1% KSR, about 10 ng/mL PI-103, about 10 ng/mL BMP4, and about 20 ng/mL FGF2 on day 2.

11. The method of claim 1 further comprising adding about 0.2% KSR and about 10 ng/mL PI-103 on day 3.

12. The method of claim 1 wherein the antibiotic is doxycycline, Gentamicin, minocycline, demeclocycline, methacycline, KB-R7943, or Ruthenium Red.

13. The method of claim 12 wherein the antibiotic is doxycycline or Gentamicin.

14. The method of claim 13 the antibiotic is doxycycline.

15. The method of claim 14 wherein the concentration of doxycycline is less than 10.0 μg/mL.

16. The method of claim 15 wherein the concentration of doxycycline is 0.1-3.0 μg/mL.

17. The method of claim 1 wherein the cell culture media further comprises Y-27632.

18. The method of claim 1 wherein the Matrigel-coated surface is the surface of a cell culture plate or a cell culture well.

19. A method of inducing differentiation of a pluripotent stem cell (PSC) to a definitive endoderm cell comprising:
(a) contacting a PSC with a Matrigel-coated surface;
(b) incubating the PSC for about seven days in RPMI media supplemented with Gem21 NeuroPlex without insulin at lx concentration, Glutagro about 2 mM, non-essential amino acids at 1× concentration, about 0.5 mM sodium butyrate, about 100 ng/mL Activin A, and 1-3 μM doxycycline;
(c) adding about 2% KnockOut Serum Replacement (KSR), about 10 ng/mL CHIR-99021, about 10 ng/mL PI-103, about 10 ng/mL BMP4,and about 20 ng/mL FGF2 on day 1;
(d) adding about 1% KSR, about 10 ng/mL PI-103, about 10 ng/mL BMP4, and about 20 ng/mL FGF2 on day 2; and
(e) adding about 0.2% KSR and about 10 ng/mL PI-103 on day 3;
wherein definitive endoderm cells are present in the culture on about day 7.

20. The method of claim 19 wherein the media is supplemented with 1 μM doxycycline.

21. A method of inducing differentiation of a definitive endoderm cell to a hepatoblast comprising:
(a) contacting a definitive endoderm cell with cell culture media comprising Matrigel, wherein the cell culture media is supplemented with Gem21 NeuroPlex without insulin, Glutagro, NEAA, monothioglycerol, human insulin and dexamethasone; and
(b) incubating the definitive endoderm cell in the cell culture media for about five days, thereby producing a hepatoblast.

22. The method of claim 21 wherein the cell culture media is Iscove's modified Dublecco's medium.

23. The method of claim 21 wherein the cell culture media comprises about ??? Gem21 NeuroPlex without insulin at 1× concentration, about 1× concentration Glutagro, about 1× concentration non-essential amino acids, about 0.3 mM monothioglycerol, about 5 μg/mL human insulin, about 100 nM dexamethasone, about 10 ng/mL Fibroblast Growth Factor 2 and about 20 ng/mL Bone Morphogenetic Protein 4.

24. A method of inducing differentiation of a hepatoblast to a hepatocyte comprising:
(a) contacting the hepatoblast for about 5 days with about 10 ng/mL Fibroblast Growth Factor 2, about 20 ng/mL Bone Morphogenetic Protein 4, and about 20 ng/mL human Hepatocyte Growth Factor; and
(b) incubating the hepatoblast in Lonza Hepatocyte Culture media BulletKit comprising about 20 ng/mL Hepatocyte Growth Factor and about 20 ng/mL Oncostatin M for about five days, wherein the Lonza Hepatocyte Culture media BulletKit lacked Epidermal Growth Factor, thereby producing a hepatocyte.
